(19) United States
(12) Patent Application Publication
(10) Pub. No.: US 2007/0196557 A1
(43) Pub. Date: Aug. 23, 2007

(54) Edible free-flowing plant-origin powder and a method for producing the same

(75) Inventors: Aharon Weissman, Misgav (IL); Eyal Ginzberg, Zichron-Yaakov (IL); Ron Frim, Haifa (IL)

Correspondence Address: FAY SHARPE LLP, 1100 SUPERIOR AVENUE, SEVENTH FLOOR, CLEVELAND, OH, 44114, US

(73) Assignee: IMI TAMI - Institute for Research and Development Ltd., Haifa (IL)

(21) Appl. No.: 11/445,645

(22) Filed: Jun. 2, 2006

(30) Foreign Application Priority Data
Feb. 20, 2006 (IL) IL 173,815

(51) Int. Cl. A23L 2/00

(52) U.S. Cl. 426/599

(57) ABSTRACT

The present invention pertains to free flowing food additives powders comprising processed fruit juice being loaded on a plant-origin solid support.

### FIELD OF THE INVENTION

The present invention generally relates to a whole “Green” edible free-flowing plant-origin powder (FFPP) containing no artificial additives and carriers. The invention also relates to methods for producing and using the same.

### BACKGROUND OF THE INVENTION

It is known in the art that nutritional compositions and fruits' ingredients, especially in grapes, citrus, apples, possess various benefits for human health apart from the well known benefits of vitamins, minerals and other nutrients considered to be the main ingredients of fruits. Apples, grapes and many other agricultural plants are hence commercially utilized a source of phenolics or plant phenolics due to their availability, high phenolics' content and respectively low market price.

It was suggested elsewhere that plant phenols inhibit mutagenesis and carcinogenesis by virtue of antioxidant or electrophile trapping mechanisms, positively effects on the human cardio-thoracic condition and act as potent modulators of arachidonic metabolism cascade pathways. Moreover, certain plant phenols can be effective inhibitors of chemical mutagens, in vitro, and/or carcinogenesis in vivo.

Encapsulated flavoring compositions are commercially required due to their particularly long shelf life. Those compositions are usually provided by emulsifying flavoring compositions in molten carbohydrate mixtures with subsequent shaping. U.S. Pat. Nos. 4,707,367; 4,499,112; 3,704,137; 3,041,180 and 2,809,895 disclose processes of encapsulating flavorings utilizing a solvent bath, which essentially evolves with a complex solvent treatment wherein the resulted particles are characterized by a low glass transition temperature (Tg).

Shaped strands can alternatively be provided in a solvent-free process by extruding and solidifying the obtained product in air. Such a procedure is disclosed in U.S. Pat. Nos. 5,603,971; 5,601,865; 5,087,461; 5,786,017; 5,009,900; and, 4,820,534. Those techniques are characterized by significant industrial drawbacks, such as increased loading with surface flavorings, and formation of harmful high fine dust content.

Moreover, it is almost impossible to transform fruit juice into a stable and flowing powder suitable for capsule filling by commercially available techniques. A suitable carrier, adapted to effectively load the natural or fermented juice to a practical concentration is required. This carrier may be selected from the contents or either intact or processed parts of fruits, plants and vegetables, such as leaves, peels, fruits, etc.

A method of producing a natural free-flowing plant powder (FFPP) having a long shelf life, which is preferably suitable for encapsulation, is desirable.

### SUMMARY OF THE INVENTION

The present invention thus pertains to free flowing food additives powders comprising processed fruit juice being loaded on a plant-origin solid support. Preferably, at least a portion of the solid support is milled oil-extracted plant solids, especially seeds, stems or roots. The oil-extracted plant solid product may comprise one or more ingredients selected from a group comprising plant seed cake and especially fruit seed cake, plant seeds and especially fruit seeds, plant seeds powder and especially fruit seeds powder. At least a portion of the fruit juice is possibly processed by a drying, squeezing, spray drying, freeze drying, extracting, fermenting and/or concentrating means. The fermented product may comprise fermented juice, fermentation mixture comprising plant peel and especially fruit peel, water, sugar and yeast or any combination thereof. The free-flowing plant-origin powder as define above may comprise about 1 to about 40% w/w of processed fruit juice. The fruit juice may comprise any fruits, plants or derivatives thereof, especially apple juice, grape juice or a mixture thereof. The plant seeds may comprise any seeds, especially apple seeds, grape seeds or a combination thereof.

It further in the scope of the present invention, wherein a natural plant-origin powder rich inter alia of phenolic contents is provided. The aforesaid FFPP is preferably yet not exclusively adapted to be utilized as a food, food ingredient, food supplement, food additive, neutraceutical, cosmetics, medicaments or pharmaceutics.

It also in the scope of the present invention wherein method of producing an edible FFPP is provided, especially wherein it comprises step or steps of loading slurry of processed fruit juice on a pre-treated plant-origin solid support. The pre-treated plant-origin solid support is possibly obtained by extracting a predetermined measure of oil from said plants' solids or milling the same. The method may comprise steps of drying pre-pressed seeds (1), pre-milling the same (2), milling (3), admixing the same in a diluted fermented plant juice (4), spray drying the obtained slurry (5) and packaging the obtained FFPP (6). The processed fruit juice is possibly provided by operations selected from a group comprising drying, squeezing, spray drying, freeze drying, extracting, fermenting and/or concentrating a predetermined plant's slurry. The fermenting process is possibly provided by utilizing fermented juice, fermentation mixture comprising plant peel and especially fruit peel, water, sugar and yeast or any combination thereof. The drying of the product is possibly provided by freeze drying methods, so as the vitality of the fruit ingredients are maintained. The method is especially but not exclusively utilizable wherein the plant is apple, tomato, or grape. The method may comprise step of drying the homogenized slurry to produce an edible free-flowing plant-origin powder. The oil-extracted plant solid product comprising one or more ingredients is preferably selected from a group comprising plant seed cake and especially fruit seed cake, plant seeds and especially fruit seeds, plant seeds powder and especially fruit seeds powder. The method may comprise step of admixing the fruit juice ingredient such that the humanized slurry comprising about 5 to about 20% w/w of processed fruit juice. The fruit juice ingredient may comprise inter alia apple juice, grape juice or a mixture thereof. The plant solids may comprise inter alia apple seeds, grape seeds or a combination thereof. The edible free-flowing plant-origin powder product is preferably rich of phenolic contents.

Alternatively, a method of producing FFPP food, food ingredient, food supplement, food additive, nutraceutical, cosmetics medicaments or pharmaceutics as defined above is provided. The method may further comprise a step of extracting plant phenolics or other compositions from macerated plants. The method may comprise steps of a. obtaining fruit juice and removing of the bulk of the solid fruit material (rape or pomace); b. fermenting; c. concentrating said juice by distillation to provide a crude concentrate; d. pre-milling the carrier solids, e.g., to about d100<0.5 mm; e. miling the same e.g., to about d50<40 μm; f. diluting the same with a fluid, e.g. about 1:10 (w/w); g. admixing liquid portion with a milled carrier; h. spray drying the same to provide a solid concentrate of plant products; and, i. packaging the same. The method may comprise steps of drying pre-pressed seeds or intact seeds (21), milling (23), mixing the same with undiluted fermented plant juice so wet powder is obtained (24), cooling (25), freeze drying the same (26), disintegrating the powder (27) and packaging (28) the FFPP.

Alternatively, the method may comprise steps selected from a group including: a. obtaining fruit juice and removing of the bulk of the solid fruit material (rape or pomace); b. fermenting the same; c. concentrating said juice by distillation to provide a crude concentrate; d. milling the carrier solids, e.g., to about d100<0.5 mm; e. admixing liquid portion with a milled carrier and freeze drying the same to provide a solid concentrate of plant products; f. disintegrating the same, e.g., by a rotating impeller means on about 1 mm screen; and, g. packaging the same.

These and other non-limiting aspects and/or objects of the development are more particularly disclosed below.

### BRIEF DESCRIPTION OF THE DRAWING AND FIGURES

In order to understand the invention and to see how it may be implemented in practice, a plurality of preferred embodiments will now be described, by way of non-limiting example only, with reference to the accompanying drawing, in which

FIG. 1 discloses a method (10) for obtaining a plant powder in a process comprising a spray dryer; and,

FIG. 2 discloses an alternative method (20) for obtaining a plant powder in a process comprising a freeze dryer.

### DESCRIPTION OF THE PREFERRED EMBODIMENTS

The following specification taken in conjunction with the drawings sets forth the preferred embodiments of the present invention. The embodiments of the invention disclosed herein are the best modes contemplated by the inventors for carrying out their invention in a commercial environment, although it should be understood that various modifications can be accomplished within the parameters of the present invention.

The terms “phenolics”, “fruit phenolics”, “polyphenols” or “plant phenolics” are used herein substantially interchangeably and in the manner normally used in food chemistry and related art. That is, these terms refer to non-toxic substances naturally occurring in plants, primarily in fruits with special emphasis on apples and grapes. The term is also denotes to edible compounds comprising in their formula a hydroxyl function attached to an aromatic carbon atom in analogy to the well known chemical phenol, or these compounds have a chemical structure closely related to or derived from a compound having an phenolics hydroxyl group.

The terms “fruit”, “vegetable”, “plant-origin” or “plant” are used hereinafter simultaneously and interchangeably to any single plant-origin matter, to a combination of a plurality (i.e., more than one) fruits, vegetables and/or plants, substantially comprising the fruit, vegetable or combination thereof, its flash or juice, any of its parts, such as its peels or skin, leaf, flower, stem, roots and/or seeds; being potentially extract, lyophilized, frozen, fermented, processed, sterilized, acidified, bleached, leached, milled, ground, steamed, dried, distilled or any combination thereof. The examples are thus related to apple and grape fruits but are by no mean limiting the scope of the process described as a general pathway for the production of powdered natural material.

For example, useful fruits, juices and carriers are selected in a non-limiting manner from deciduous plants and fruits, citrus fruits, tomato juice, carrot juice, fermented juice from fruit, vegetable or other plant source.

The term ‘nutrichemical’ refers hereinafter to “medical food”, “optimum food”, “phytochemical”, “phytomin”, “phytonutrient”, “therapeutic food” and to any food containing naturally occurring compounds that (a) are customarily considered neither nutrients nor toxins and (b) may contribute to preventing or curing disease.

The term ‘phenolic content’ refers hereinafter in a non-limiting manner to any composition or mixtures thereof selected from a group comprising cyaniding, delphinidin, caffeic acid, chlorogenic acid, gallic acid, ellagic acid, luteotin, quercetin, kaempferol, naringenin, coumesterol, 17-alpha-esterol, esterone, estriol, testosterone, beta-sitosterol, coumesterol, gamma-tocopherol, punicic acid, campesterol, stigmasterol or a combination thereof.

The term ‘solids’ refers hereinafter in a non-limiting manner to any suitable carrier adapted to carry the dried liquids, and is selected inter alia from organic matter, e.g., natural products, especially plant origin materials, e.g., seeds, stems, roots etc, polysaccharides, proteins, edible polymers, e.g., poly lactic acids, nylon 12 etc, biocompatible inorganic matter, e.g., magnesia, silicates, or a mixture thereof.

A FFPP is hereto provided. This edible ‘running’ powder is natural (i.e., the plant source was not chemically and physically intact) and rich with the minerals, vitamins, natural phenolics and other nutrients of the fruit or vegetable raw material. The powder is preferably utilized for encapsulation as a supplement or additive in various food products, cosmetics and nutraceuticals. This powder is rich in natural minerals, vitamins natural phenolics and other nutrients of the fruit or vegetable and which does not impart an unnatural or undesired organolepic quality (taste, texture, flavour etc) of the fruit or vegetable which was used.

The disclosed process utilizes a plant-origin carrier to be loaded by fruit juice. The loading process comprises at least one step selected from a group of coating, loading, impregnating, doping, immobilizing, interacting, at least partially solubilizing or dispersing, crystallizing, precipitating or admixing means. Pressed fruit seeds, pits, pips or their like are useful carriers. The particle size distribution of the pressed seeds is adjustable such that a preferred size distribution is provided by milling the seeds; forming wet powder or suspension of the same, and finally drying it (e.g., freeze drying) to a powder. The carrier the juice may be obtained from different fruits or plants or at least partially obtained from the same source. Alternatively, the carrier the juice may be obtained from the same fruits or plants source or at least partially obtained from a different source. FFPPs obtained by freeze-drying methods may be regarded by some as healthier than others.

It is in the scope of the present invention wherein the phenolics utilized thereof are isolated from fruits, primarily yet not exclusively from apples, grapes, their parts thereof and their products; and comprising in a non-limiting manner any of the group of gallic acid, flavan-3-ols, phloridzin, cinnamates, hydroxymethyl furfural and anthocyanins, phenolic acids and especially derivatives of benzoic acid and derivatives of cinnamic acid, stilbenes, lignans, flavonoids, flavones, isoflavones, flavanones, anthocyanidins, and flavanols (catechins and proanthocyanidins for example), hydroxybenzoic acid, hydrolyzable tannins, hydroxycinnamic acids (such as p-coumaric, caffeic, ferulic, and sinapic acids), glycosylated derivatives or esters of quinic acid, shikimic acid, and tartaric acid, chlorogenic acid etc.

In accordance with one embodiment of the present invention to disclose an industrial process for the production of free-flowing plant-origin powder. The powder is composed inter alia of fruit fermented juice (FJ) loaded on milled solids (MS). The concentration of the dry FJ in the end product ranges from about 1 to 40% w/w. The powder is stable and free flowing and is adapted to be used for capsulation in tablets, pills, capsules etc. The feasibility of the process was demonstrated on a 2 kg bench scale and on a 100 kg pilot scale.

A spry dry-based method of transforming FJ into a stable and flowing powder suitable for capsule filling is hereto disclosed. The method comprises inter alia at least two stages of milling of vegetative solids e.g., seeds. The first pre-milling is provided by a means e.g., of rotor beater mill (Retcsh, Germany); followed by a milling step, e.g., by utilizing vortex milling means (Super Fine Israel); forming a suspension comprising the same and a fermented juice; and then drying it to a free-flowing powder. The method defined above is especially useful in unit operation comprising a spray dryer.

Raw materials for the process comprising fruit solids (i.e., the carrier) and fermented juice (i.e., the loaded liquid). The carrier is pre-pressed fruit solids (PPS) which contain about half of the initial oil content, e.g., about 15 to 20% wlw. Solids with different oil concentrations, such as 0% w/w oil (e.g., pressed and extracted seeds) and 100% w/w/oil (e.g., whole seeds) were tested as well. It is in the scope of the present invention and in a non-limiting manner, FJ loaded liquid comprises about 22% solids (loss on drying, LOD), without ethanol; and about 15% solids (LOD), optionally with about 4 to 9% w/w ethanol. MPPS denotes milled (-pressed) fruit solids.

It is according to one embodiment of the present invention to provide a freeze dry-based method, wherein plant phenolics are extracted from macerated fruits and vegetables, particularly yet not exclusively from apple peels and apple cores, grapes and grape rape (pomace) etc.

Said freeze dry-based process further comprising:
a. obtaining fruit juice and removing of the bulk of the solid fruit material (rape or pomace);
b. fermenting;
c. concentrating the solvent by distillation to provide a crude concentrate;
d. milling the carrier solids, e.g., to about d₁₀₀<05 mm.
e. admixing liquid portion with a milled carrier and freeze drying the same to provide a solid concentrate of plant products;
f. disintegrating the same, e.g., by a rotating impeller means on about 1 mm screen;
g. if required, utilizing solid portion as food supplements or encapsulating the same.

It is acknowledged in this respect that the fermentation is an optional step and concentration of juice (not fermented juice) is also applicable in various systems.

It is according to yet another embodiment of the present invention wherein the process of producing a free-flowing plant-origin powder, which is natural and rich with minerals, vitamins, plant phenolics and other nutrients of the fruit or vegetable raw material.

It is acknowledged in this respect that according to one embodiment of the present invention, two milling steps are required in a process with a spray dryer and according to another embodiment; one milling step is required in a process comprising freeze dryer.

It is according to still another embodiment of the present invention wherein the process of producing a plant-origin powder; this method comprises in a non-limiting manner the following steps.

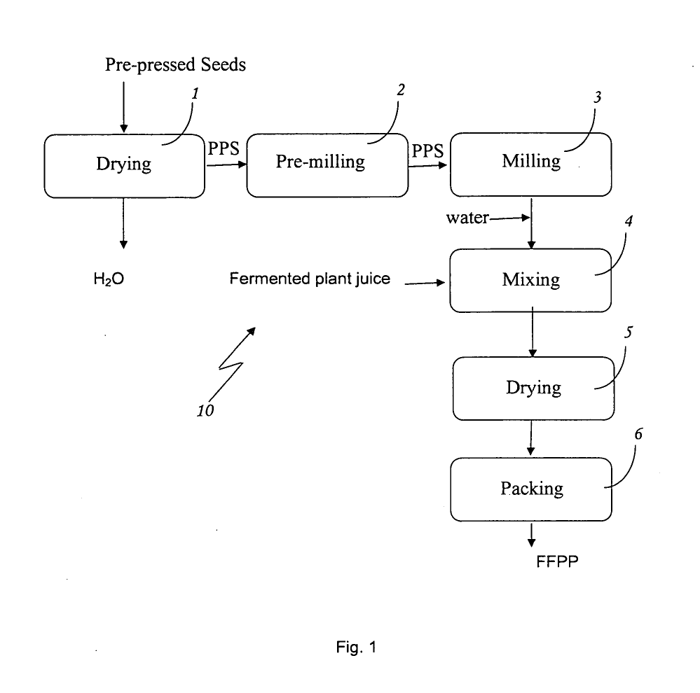

Reference is now made to FIG. 1, presenting a method (10) for obtaining a plant powder in a process comprising a spray dryer. The method comprising the steps of drying pre-pressed seeds (1), pre-milling the same (2), milling (3), admixing the same in a fermented plant juice (4), spray drying the obtained slurry (5) and packaging the same (6) so as a fruit-originated running powder is obtained. More specifically, pressed fruit seeds comprising from 0% to.about 50% (wt) oil, e.g., 7.5% (wt) oil are dried in a dryer, a double cone dryer for example. The dried pressed fruit seeds is pre-milled in a suitable mill, impact mill for example such that dried milled seeds is characterized e.g., by d₁₀₀<0.5 mm is obtained. In a subsequent step or steps of milling, e.g., by a means of a vortex mill, fine powder is obtained, e.g., d₅₀ is laying in the range of about 10 μm to about 40 μm. Effective measure of fluids, e.g., treated water, natural or fermented juice, plant juice comprising about 15% (wt) solids on drying or a mixture thereof are admixed to the powder in a mixer, e.g., high shear mixer, so as a suspension comprising about 5 to about 10% (wt) solids and about 7.5% to about 40% dried juice is obtained in the end product. The suspension in then dried in a spray dryer. The dried suspension, LOD<1.0%, d₉₀<0.5 mm is obtained. The final fruit layered product may then be packed.

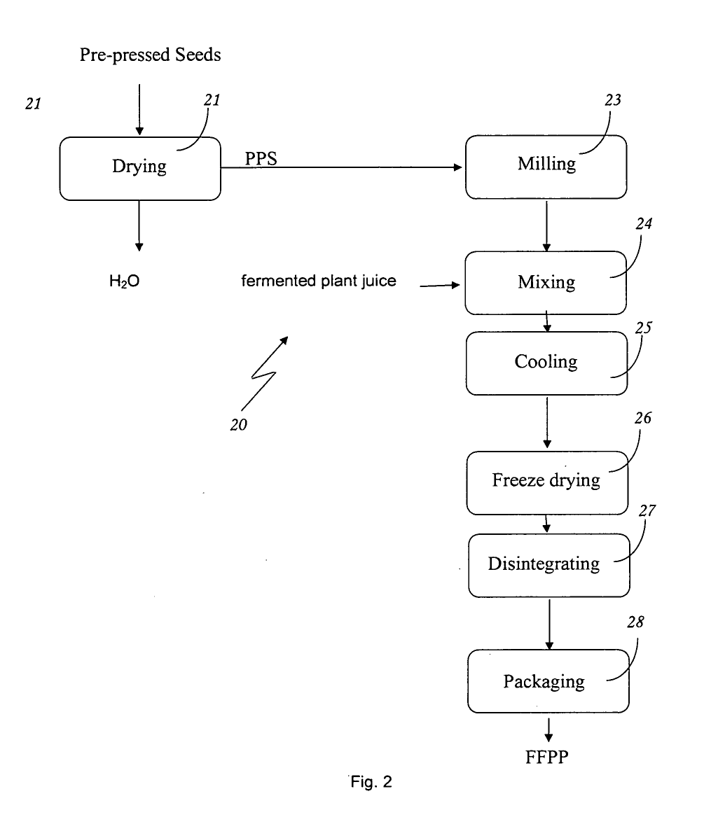

Reference is now made to FIG. 2, presenting an alternative method (20) for obtaining a plant layered product in a process comprising a freeze dryer. The method comprising steps of drying pre-pressed seeds and/or pre-fermented seeds (21), milling (23), mixing the same with fermented plant juice so as wet powder is obtained (24), cooling (25), freeze drying the same (26), disintegrating the powder (27) and packaging (28) the plant fruit-originated running powder product. More specifically, pressed fruit seeds as defined above is dried in a dryer, a double cone dryer for example. The dried pressed fruit seeds (LOD<1%) is milled, e.g., by a means of an impact mill, and a powder of d₁₀₀<0.5 mm is obtained. Effective measure of fluids, e.g., treated water, natural or fermented juice, plant juice comprising about 8 to about 15% (wt) solids on drying or a mixture thereof are admixed to the powder in a mixer, e.g., ribbon mixer, so as a wet powder comprising about 54% (wt) solids and about 7.5% to about 14% dried juice may be obtained. Moreover, higher values to 30% may are obtainable in various systems. The wet matter in then cooled batch wise or continuously to about −18° C. Subsequently, the frozen powder is applied in a freeze dryer, so as dry layered powder, LOD<1.0% is obtained. The fruit layered product is disintegrated if needed and a final product powder (d₉₀<0.5 mm) may then be packed.

Table 1 below shortly summarizes few of the differences between the aforesaid spray-dry and freeze dry methods of the present invention.

TABLE 1
Freeze dry vs Spray dry Processes

| | Spray Dry | Freeze Dry |
|---|---|---|
| Plant-origin solid | Two-stages: | Single stage: |
| support | (i) pre-milling d₁₀₀ < 0.5 | (i) milling d₁₀₀ < 0.5 mm |
| | mm; | |
| | (ii) milling d₅₀ < 40 μm | |
| Processed fruit | Optionally, concentrated | Concentrated fermented |
| juice | fermented juice | juice e.g., 10% (w/w) |
| | e.g., 15–20% (w/w) | |
| Formulation | 1 to 40% load of juice on | At least 10% load of |
| | solid support | juice on solid support |
| Dilution by | Required | Not required |
| admixed water | | |
| Feed to dryer | Suspension, | Wet powder, |
| | 5% solids (w/w) | at least 50% solids (w/w) |
| Disintegration | Not required | Required |
| before packaging | | |
| Flavonoids | About 20% of initial | About 100% of initial |
| content in final | concentration | concentration |
| product* | | |

*Flavonoids content in final product is an antioxidant, provided herein as an indicator for product vitality.

The development will further be illustrated in the following non-limiting example. This example is intended to be illustrative only and the disclosure is not intended to be limited to the materials, conditions, process parameters and the like recited herein. All proportions are by weight unless otherwise indicated.

### EXAMPLE

A freeze-dried method as generally defined above, for obtaining an antioxidant-rich powder is hereto disclosed. Rape of dark (red) grapes is dried in double cone at 80° C. and about 60 to 100 mbar for 12 hours to LOD of about 3%, milled to d₁₀₀<0.5 mm and loaded with concentrated red fermented grape juice (wine) (about 9% solids). The wet powder of about 54% solids (wt/wt) is dried in freeze drying and later disintegrated to d₁₀₀<05 mm give a stable and free flowing powder comprising about 80 to 100% of the initial antioxidant concentration suitable for capsules filling.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A biocompatible free-flowing plant-origin powder (FFPP), comprising processed fruit juice being loaded on a plant-origin solid support.

2. The powder according to claim 1, wherein at least a portion of the solid support is milled oil-extracted plant solids, seeds, stems or roots.

3. The product of claim 1, wherein said oil-extracted plant solid product comprises one or more ingredients selected from a group consisting of plant seed cake, fruit seed cake, plant seeds, fruit seeds, plant seeds powder and fruit seeds powder.

4. The powder according to claim 1, wherein at least a portion of the fruit juice is processed by a drying, squeezing, spray drying, freeze drying, extracting, fermenting or concentrating means.

5. The product of claim 4, wherein the fermented product comprises fermented juice or a fermentation mixture comprising plant peel, water, sugar and yeast, or any combination thereof.

6. The free-flowing plant-origin powder according to claim 1, comprising from about 1 to about 40% w/w of processed fruit juice.

7. The powder according to claim 1, wherein the fruit juice comprises any fruits, plants or derivatives thereof.

8. The powder according to claim 1, wherein plant seeds comprise apple seeds, grape seeds or a combination thereof.

9. A natural plant-origin powder rich inter alia of phenolic contents.

10. An FFPP according to claim 1, adapted to be utilized as a food, food ingredient, food supplement, food additive, nutraceutical, cosmetics, medicaments or pharmaceutics.

11. A method of producing an edible FFPP, comprising loading a slurry of processed fruit juice on a pre-treated plant-origin solid support.

12. The method according to claim 11, wherein the pre-treated plant-origin solid support is obtained by extracting a predetermined measure of oil from said plants' solids or milling the same.

13. The method according to claim 11, comprising the steps of drying pre-pressed seeds (1), pre-milling the same (2), milling (3), admixing the same in a diluted fermented plant juice (4), spray drying the obtained slurry (5) and packaging the obtained FFPP (6).

14. The method according to claim 11, wherein the processed fruit juice is provided by operations selected from a group comprising drying, squeezing, spray drying, freeze drying, extracting, fermenting and/or concentrating a predetermined plant's slurry.

15. The method according to claim 14, wherein the fermenting process is provided by utilizing fermented juice or a fermentation mixture comprising plant peel, water, sugar and yeast, or any combination thereof.

16. The method according to claim 14, wherein the drying of the product is provided by freeze drying methods, so as the vitality of the fruit ingredients is maintained.

17. The method according to claim 11, wherein the plant is apple or grape.

18. The method according to claim 11, additionally comprising drying the homogenized slurry to produce an edible free-flowing plant-origin powder.

19. The method according to claim 11, wherein the oil-extracted plant solid product comprises one or more ingredients selected from a group consisting of plant seed cake, fruit seed cake, plant seeds, fruit seeds, plant seeds powder, and fruit seeds powder.

20. The method according to claim 11, comprising admixing the fruit juice ingredient such that the humanized slurry comprising from about 5 to about 20% wlw of processed fruit juice.

21. The method according to claim 11, wherein the fruit juice ingredient comprises apple juice, grape juice or a mixture thereof.

22. The method according to claim 11, wherein plant solids comprise apple seeds, grape seeds or a combination thereof.

23. The method according to claim 11, wherein the edible free-flowing plant-origin powder product is rich of phenolic contents.

24. A method of producing FFPP food, food ingredient, food supplement, food additive, nutraceutical, cosmetics medicaments or pharmaceutics according to claim 11.

25. The method according to claim 11, additionally comprising extracting plant phenolics or other compositions from macerated plants, comprising:
a. obtaining fruit juice and removing of a portion of the solid fruit material (rape or pomace);
b. fermenting;
c. concentrating said juice by distillation to provide a crude concentrate;
d. pre-milling the carrier solids;
e. milling the same;
f. diluting the same with a fluid;
g. admixing liquid portion with a milled carrier; and,
h. spray drying the same to provide a solid concentrate of plant products.

26. The method according to claim 11, comprising the steps of drying pre-pressed seeds or intact seeds, milling, mixing the same with undiluted fermented plant juice so wet powder is obtained, cooling, freeze drying the same, disintegrating the powder and packaging the FFPP.

27. The method according to claim 11, comprising the steps of:
a. obtaining fruit juice and removing the bulk of the solid fruit material (rape or pomace);
b. fermenting the same;
c. concentrating said juice by distillation to provide a crude concentrate; p1 d. milling the carrier solids;
e. admixing liquid portion with a milled carrier and freeze drying the same to provide a solid concentrate of plant products; and,
f. disintegrating the same.
